(19) United States
(12) Patent Application Publication
(10) Pub. No.: US 2011/0071302 A1
(43) Pub. Date: Mar. 24, 2011

(54) PROCESS FOR PREPARING INTERMEDIATE COMPOUND FOR SYNTHESIZING AN ANTIULCERANT

(75) Inventors: Dong Yeon Kim, Seoul (KR); Jun Yeoun Lee, Suwon (KR); Kwi Hyung Cho, Hwaseong (KR); Sung Tae Park, Yongin (KR); Jung Woo Kim, Gyeonggi Do (KR); Doo Hyuk Pyun, Yongin (KR); Sang Don Nam, Hwaseong (KR); Hee Yun Kim, Yongin (KR)

(73) Assignee: IL-YANG PHARM. CO., LTD., Yongin, Gyeonggi-Do (KR)

(21) Appl. No.: 12/993,086

(22) PCT Filed: Nov. 20, 2008
(86) PCT No.: PCT/KR2008/006849

§ 371 Date: Nov. 16, 2010

(30) Foreign Application Priority Data
Jun. 12, 2008 (KR) 10-2008-0055111

(51) Int. Cl. C07D 403/10

(52) U.S. Cl. 548/306.1

(57) ABSTRACT

The present invention relates a novel method of preparing an intermediate which is useful for synthesizing an antiulcerant. The present invention provides a method of preparing an intermediate of an antiulcerant which can obtain a high purity compound in high yield, with reduced production cost/time as compared to a conventional method.

### TECHNICAL FIELD

The present invention relates a novel method of preparing an intermediate which is useful for synthesizing an antiulcerant.

### BACKGROUND ART

A gastric/duodenal ulcer is a digestive disease caused by various factors such as mental stress, eating habits, intake of spicy food, etc. and is primarily caused by gastric mucous membrane damage due to hyperacidity. Therapeutic agents of the gastric/duodenal ulcer include an antacid for neutralizing gastric acid, an antipepsin agent, a gastric mucous membrane protecting agent, an anticholinergic agent for inhibiting gastric acid secretion, a parasympatholytic agent, a gastric mucous membrane protecting agent, an H₂ receptor antagonist, etc. However, since conventional gastric ulcer therapeutic agents, such as an antacid, and a central nervous system agent, have an unsatisfactory effect and may cause side effects after long-term administration, the use of an H₂ receptor antagonist, that is, a gastric/duodenal ulcer therapeutic agent having a new working mechanism, has been recently increased.

Also, PPI agents such as omeprazole have been made into various types of formulations and have been widely used because their anti-ulcer effect was proved to be much greater than that of conventional H₂ receptor antagonists, such as cimetidine, famotidine, ranitidine, etc. Meanwhile, inventors of the present invention invented ilaprazole, which is a compound with reduced side effects and improved therapeutic effects, as compared to a conventional PPI compound, through a long time research for developing a novel PPI compound. The invention was patent-registered in Korea (Korea Patent No. 179401) and foreign countries. Reaction Scheme 1 illustrates a general preparation method of ilaprazole.

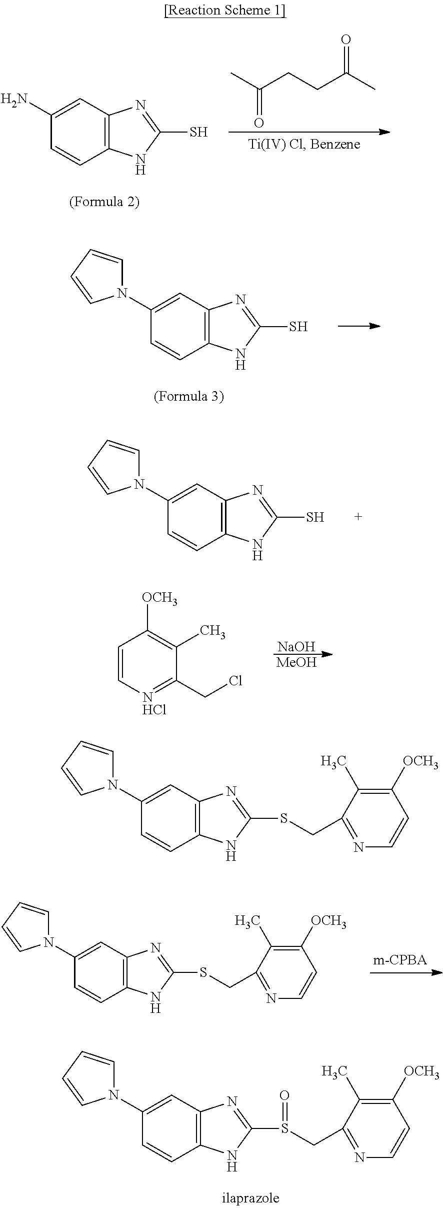

The Reaction Scheme 1 illustrates a method of preparing 5-(1H-pyrrole-1-yl)-2-mercaptobenzimidazole, that is, a compound represented by Formula 3, the method including the steps of: adding 2-mercapto-5-aminobenzimidazole (100 g, 0.61 mole) represented by Formula 2, tetrahydrofuran (1200 ml) and succinaldehyde (57.34 g, 0.67 mole), followed by cooling to 10° C. or less; adding a titanium chloride (11.57 g, 0.06 mole) solution dissolved in tetrahydrofuran (200 ml); stirring the mixture at 60° C. for 15 hours and adding water; and carrying out crystallization after layer-separation. Meanwhile, a conventional preparation method has a disadvantage in that, due to low yield (about 21%) and low purity, a large amount of by-products is generated in the following reaction and the reaction time is too long. In addition, succinaldehyde used for the method is expensive, thereby increasing production costs.

### DISCLOSURE OF INVENTION

### Technical Problem

Accordingly, the present invention has been made to solve the above-mentioned problems occurring in the prior art, and the present invention provides a method of preparing a compound represented by Formula 3, which can obtain a high purity compound in high yield, with reduced production cost/time as compared to a conventional method.

### Technical Solution

The present invention provides a method of preparing the compound represented by Formula 3 (that is, an intermediate of an antiulcerant) through a reaction of the compound represented by Formula 1 with the compound represented by Formula 2 (that is, 2-mercapto-5-aminobenzimidazole).

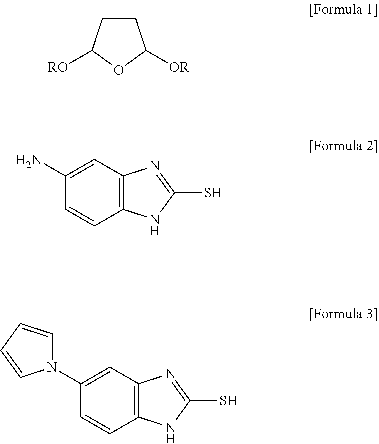

In the above Formula, R represents C₁₋₆ alkyl.

According to an embodiment of the present invention, the preparation method includes the steps of: cyclizing the compound represented by Formula 1 and the compound represented by Formula 2 (2-mercapto-5-aminobenzimidazole) with acid and a reaction solvent; separating an organic layer after neutralization by adding a base aqueous solution; and crystallizing the compound represented by Formula 3 by using a crystallization solvent after drying and concentrating the organic layer.

According to another embodiment of the present invention, the preparation method may further include the step of adding an extractant to the resultant product, after the cyclizing step.

According to a further embodiment of the present invention, the preparation method includes the steps of: cyclizing the compound represented by Formula 1 and the compound represented by Formula 2 (2-mercapto-5-aminobenzimidazole) by adding acid and a reaction solvent thereto and stirring; adding an extractant to the resultant product, and separating an organic layer after neutralization by adding a base aqueous solution; and drying/concentrating the separated organic layer by using a drying agent and crystallizing a final compound by using a crystallization solvent.

In the present invention, the acid that may be used in the cyclizing step may include: at least one material selected from the group including sulfonic acid, phosphoric acid, nitric acid, perchloric acid, formic acid, acetic acid, propionic acid, succinic acid, gluconic acid, p-hydroxybenzoic acid, salicylic acid, methanesulfonic acid, ethanesulfonic acid, hydroxyethanesulfonic acid, ethylene sulfonic acid, toluene sulfonic acid, naphthyl sulfonic acid, sulfanilic acid, camphorsulfonic acid, quinic acid, o-methylenemandelic acid, hydrogen benzene sulfonic acid and tartaric acid; preferably at least one material selected from the group including sulfonic acid, phosphoric acid, nitric acid, perchloric acid, formic acid, acetic acid, propionic acid, succinic acid, gluconic acid, p-hydroxybenzoic acid, salicylic acid and methanesulfonic acid; and more preferably acetic acid or toluene sulfonic acid.

In the present invention, the reaction solvent that may be used in the cyclizing step may be selected: from the group including water, xylene, toluene, tetrahydrofuran, 1,2-dichloroethane, lower alkanol, acetone, ether, dichloromethane, acetonitrile, dimethylsulfoxide, dimethylformamide and a mixture thereof; preferably from the group including water, xylene, toluene, tetrahydrofuran, 1,2-dichloroethane, lower alkanol, acetone and a mixture thereof; and more preferably from the group including water, xylene, tetrahydrofuran, 1,2-dichloroethane and a mixture thereof.

In the present invention, in the cyclization reaction, the temperature is not particularly limited, but mixtures may be stirred at 0 to 150° C., preferably at 0 to 80° C., and more preferably at room temperature to 80° C. Also, the stirring time is not particularly limited, but may preferably range from 1 to 10 hours.

In the present invention, in the cyclization reaction, a buffering agent, such as anhydrous sodium acetate, may be additionally used.

In the present invention, after the cyclization reaction, the resultant product may be additionally cooled. Herein, the cooling temperature is not particularly limited, but may range from −15 to 50° C., preferably from −15 to 30° C., more preferably from 0 to room temperature, and may be most preferably at 5° C.

In the present invention, the extractant that may be used in an extraction step may include: at least one selected from the group including tetrahydrofuran, 1,2-dichloroethane, lower alkanol, acetone, chloroform, dichloromethane and ethyl acetate; preferably at least one selected from the group including tetrahydrofuran and 1,2-dichloroethane; and more preferably tetrahydrofuran.

In the present invention, the base aqueous solution that may be used in a neutralization and/or layer-separation step may include: at least one selected from the group including a sodium hydroxide aqueous solution, a potassium hydroxide aqueous solution, a potassium carbonate aqueous solution, a calcium carbonate aqueous solution, a sodium methoxide aqueous solution, a sodium hydrogen carbonate aqueous solution, a pyridine aqueous solution, ammonia water, a triethylamine aqueous solution and ethyl diisopropyl amine aqueous solution; preferably at least one selected from the group including a sodium hydroxide aqueous solution, a potassium hydroxide aqueous solution, a potassium carbonate aqueous solution and a calcium carbonate aqueous solution; and more preferably a sodium hydroxide aqueous solution.

The drying agent that may be used in the present invention is not particularly limited, but may be at least one material selected from the group including anhydrous magnesium sulfate and anhydrous sodium sulfate.

The crystallization solvent that may be used in the present invention is not particularly limited, but may be a material selected from the group including n-hexane, n-heptane, ethyl acetate, tetrahydrofuran, ether, dichloromethane, chloroform, acetone and a mixture thereof, and preferably a material selected from the group including n-hexane, ethyl acetate and a mixture thereof.

In the present invention, in Formula 1, R may represent C₁₋₆ alkyl, e.g., methyl, ethyl, propyl, isopropyl, butyl, pentyl, and hexyl, preferably methyl, ethyl or propyl, more preferably methyl or ethyl, and most preferably methyl.

A method of preparing an intermediate (the compound represented by Formula 3) of an antiulcerant according to the present invention is shown in Reaction Scheme 2.

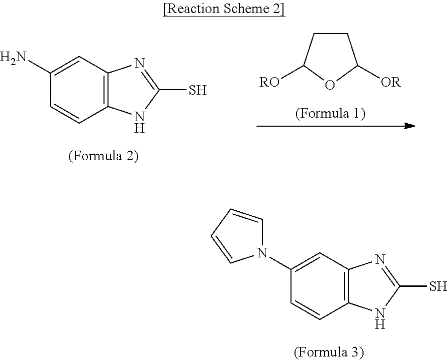

The compound represented by Formula 1 is commercially available. However, until now, in synthesis of an antiulcerant, the compound represented by Formula 1 was not prepared into the compound represented by Formula 3 through a reaction with the compound represented by Formula 2. Herein, R in Formula 1 represents C₁₋₆ alkyl.

### ADVANTAGEOUS EFFECTS

The present invention provides a method of preparing an intermediate of an antiulcerant which can obtain a high purity compound in high yield, with reduced production cost/time as compared to a conventional method.

### BEST MODE FOR CARRYING OUT THE INVENTION

Hereinafter, the present invention will be described in detail with reference to following Examples. However, the following examples are illustrative only, and the scope of the present invention is not limited thereto.

### Example 1

Water (100 ml) and anhydrous sodium acetate (16.02 g, 0.20 mole) were added, and then 2-mercapto-5-aminobenzimidazole (32.27 g, 0.20 mole), 2,5-dimethoxytetrahydrofuran (28.4 g, 0.21 mole), and acetic acid (100 ml) were added. Then, stirring was carried out at 50° C. for 4 hours. The resultant product was cooled to 5° C. and tetrahydrofuran (420 ml) was added thereto. The mixture was neutralized with a sodium hydroxide aqueous solution, and water (130 ml) was added to carry out layer-separation. Then, an organic layer was washed with a sodium hydroxide aqueous solution. The organic layer was dried by anhydrous magnesium sulfate and concentrated, and then crystallized by ethyl acetate and n-hexane to provide a final compound, that is, 5-(1H-pyrrol-1-yl)-2-mercaptobenzimidazole represented by Formula 3. Then, the obtained compound was confirmed.

M.P. 311.8° C. Direct inlet MS (EI) for C₁₁H₉N₃S m/z (relative intensity) 215 (M⁺, 100)

¹H NMR (200 MHz, DMSO) δ 6.22 (t, 2H), 7.19 (m, 3H), 7.25 (t, 2H), 12.46 (b, 1H)

Yield: 35.7 g (85%)

### Example 2

2-mercapto-5-aminobenzimidazole (20 g, 0.12 mole), 2,5-dimethoxytetrahydrofuran (15.9 g, 0.12 mole) and acetic acid (60 ml) were added and stirred at 60° C. for 5 hours. The resultant product was cooled to 5° C., and water (150 ml) and tetrahydrofuran (300 ml) were added thereto. Then, the mixture was neutralized with a sodium hydroxide aqueous solution. After layer separation, an organic layer was washed with a sodium hydroxide aqueous solution. The organic layer was dried by anhydrous magnesium sulfate and concentrated, and then crystallized by ethyl acetate and n-hexane to provide a final compound.

Yield: 15.5 g (60%)

### Example 3

2-mercapto-5-aminobenzimidazole (20 g, 0.12 mole), 2,5-dimethoxytetrahydrofuran (15.9 g, 0.12 mole), acetic acid (60 ml), and anhydrous sodium acetate (9.8 g, 0.12 mole) were added and stirred at 60° C. for 4 hours. The resultant product was cooled to 5° C., and water (150 ml) and tetrahydrofuran (300 ml) were added thereto. Then, the mixture was neutralized with a sodium hydroxide aqueous solution. After layer separation, an organic layer was washed with a sodium hydroxide aqueous solution. The organic layer was dried by anhydrous magnesium sulfate and concentrated, and then crystallized by ethyl acetate and n-hexane to provide a final compound.

Yield: 18.07 g (70%)

### Example 4

2-mercapto-5-aminobenzimidazole (20 g, 0.12 mole), 2,5-dimethoxytetrahydrofuran (15.9 g, 0.12 mole) and acetic acid (60 ml) were added, and then water (120 ml) and 1,2-dichloroethane (180 ml) were added. Then, stirring was carried out at 60° C. for 4 hours. The resultant product was concentrated and cooled to 5° C., and tetrahydrofuran (300 ml) was added thereto. Then, the mixture was neutralized with a sodium hydroxide aqueous solution, and water (150 ml) was added to carry out layer-separation. An organic layer was washed with a sodium hydroxide aqueous solution. The organic layer was dried by anhydrous magnesium sulfate and concentrated, and then crystallized by ethyl acetate and n-hexane to provide a final compound.

Yield: 17.3 g (67%)

### Example 5

2-mercapto-5-aminobenzimidazole (20 g, 0.12 mole), 2,5-dimethoxytetrahydrofuran (15.9 g, 0.12 mole), and water (240 ml) were added and stirred at 60° C. for 6 hours. The resultant product was cooled to room temperature and tetrahydrofuran (300 ml) was added thereto. After layer separation, an organic layer was dried by anhydrous magnesium sulfate and concentrated, and then crystallized by ethyl acetate and n-hexane to provide a final compound.

Yield: 13.4 g (52%)

### Example 6

2-mercapto-5-aminobenzimidazole (20 g, 0.12 mole) and 2,5-dimethoxytetrahydrofuran (15.9 g, 0.12 mole) were added, and then water (180 ml) and 1,2-dichloroethane (180 ml) were added. Then, stirring was carried out at 60° C. for 6 hours. The resultant product was concentrated and cooled to room temperature, and tetrahydrofuran (300 ml) was added thereto. After layer-separation, an organic layer was dried by anhydrous magnesium sulfate and concentrated, and then crystallized by ethyl acetate and n-hexane to provide a final compound.

Yield: 17.8 g (69%)

### Example 7

2-mercapto-5-aminobenzimidazole (30 g, 0.18 mole), anhydrous sodium acetate (14.9 g, 0.18 mole) were added, and then acetic acid (90 ml), water (180 ml), tetrahydrofuran (135 ml) and 2,5-dimethoxyhydrofuran (47.9 g, 0.36 mole) were added. Then, stirring was carried out at 60° C. for 7 hours. The resultant product was cooled to 5° C., and neutralized with a sodium hydroxide aqueous solution. After layer-separation, an organic layer was washed with a sodium hydroxide aqueous solution. The organic layer was dried by anhydrous magnesium sulfate and concentrated, and then crystallized by ethyl acetate and n-hexane to provide a final compound.

Yield: 24.62 g (63%)

### Example 8

Water (100 ml) and anhydrous sodium acetate (16.02 g, 0.19 mole) were added, and 2-mercapto-5-aminobenzimidazole (32.27 g, 0.19 mole), 2,5-diethoxytetrahydrofuran (28.4 g, 0.21 mole), and acetic acid (100 ml) were added. Then, stirring was carried out at 50° C. for 5 hours. The resultant product was cooled to 5° C., and tetrahydrofuran (300 ml) was added thereto. Then, the mixture was neutralized with a sodium hydroxide aqueous solution, and water (130 ml) was added to carry out layer-separation. An organic layer was washed with a sodium hydroxide aqueous solution. The organic layer was dried by anhydrous magnesium sulfate and concentrated, and then crystallized by ethyl acetate and n-hexane to provide a final compound.

Yield: 29.4 g (70%)

### Example 9

2-mercapto-5-aminobenzimidazole (15 g, 0.09 mole), 1,2-dichloroethane (150 ml), and water (150 ml) were added, and then 2,5-dimethoxytetrahydrofuran (13.2 g, 0.1 mole), paratoluene sulfonic acid (5.18 g, 0.03 mole), and tetrahydrofuran (100 ml) were added. Then, stirring was carried out at 60° C. for 5 hours. The resultant product was cooled to 5° C., and neutralized with a sodium hydroxide aqueous solution. After layer-separation, an organic layer was washed with a sodium hydroxide aqueous solution. The organic layer was dried by anhydrous magnesium sulfate and concentrated, and then crystallized by ethyl acetate and n-hexane to provide a final compound.

Yield: 10.16 g (52%)

### Example 10

Xylene (100 ml) and anhydrous sodium acetate (16.02 g, 0.20 mole) were added, and 2-mercapto-5-aminobenzimidazole (32.27 g, 0.20 mole), 2,5-dimethoxytetrahydrofuran (28.4 g, 0.21 mole) and acetic acid (100 ml) were added. Then, stirring was carried out at 150° C. for 2 hours. The resultant product was concentrated and cooled to 50° C., and water (240 ml) and tetrahydrofuran (420 ml) were added thereto. Then, the mixture was neutralized with a sodium hydroxide aqueous solution. After layer-separation, an organic layer was washed with a sodium hydroxide aqueous solution. The organic layer was dried by anhydrous magnesium sulfate and concentrated, and then crystallized by ethyl acetate and n-hexane to provide a final compound.

Yield: 14.7 g (35%)

### Example 11

Water (100 ml) and anhydrous sodium acetate (16.02 g, 0.20 mole) were added, and 2-mercapto-5-aminobenzimidazole (32.27 g, 0.20 mole), 2,5-dimethoxytetrahydrofuran (28.4 g, 0.21 mole), and acetic acid (100 ml) were added. Then, stirring was carried out at 10° C. for 10 hours. The resultant product was cooled to −15° C., and tetrahydrofuran (420 ml) was added thereto. Then, the mixture was neutralized with a sodium hydroxide aqueous solution, and water (130 ml) was added to carry out layer-separation. An organic layer was washed with a sodium hydroxide aqueous solution. The organic layer was dried by anhydrous magnesium sulfate and concentrated, and then crystallized by ethyl acetate and n-hexane to provide a final compound.

Yield: 22.3 g (53%)

Although an exemplary embodiment of the present invention has been described for illustrative purposes, those skilled in the art will appreciate that various modifications, additions and substitutions are possible, without departing from the scope and spirit of the invention as disclosed in the accompanying claims.

1. A method of preparing 5-(1H-pyrrole-1-yl)-2-mercaptobenzimidazole represented by Formula 3, by a reaction of a compound represented by Formula 1 with a compound represented by Formula 2, that is, 2-mercapto-5-aminobenzimidazole: 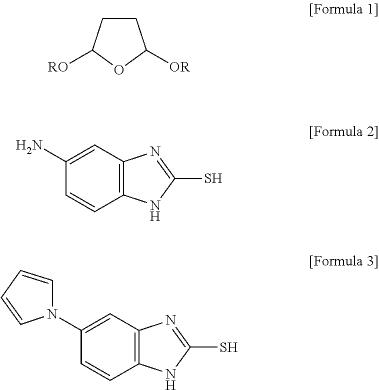
wherein R represents C₁₋₆ alkyl.

2. A method of preparing a compound represented by Formula 3, the method comprising the steps of:
carrying out cyclization of a compound represented by Formula 1 and a compound represented by Formula 2 (2-mercapto-5-aminobenzimidazole) with acid and a reaction solvent;
separating an organic layer after neutralization by adding a base aqueous solution; and
crystallizing the compound represented by Formula 3 by using a crystallization solvent after drying and concentrating the organic layer, 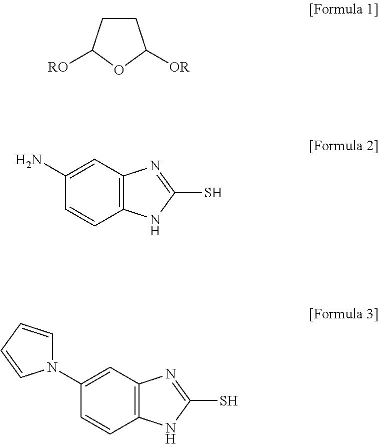
wherein R represents C₁₋₆ alkyl.

3. The method as claimed in claim 2, further comprising the step of adding an extractant to a resultant product after the cyclization.

4. The method as claimed in claim 3, wherein the extractant is at least one selected from the group including tetrahydrofuran, 1,2-dichloroethane, lower alkanol, acetone, chloroform, dichloromethane and ethyl acetate.

5. The method as claimed in claim 2 or 3, wherein the acid is at least one selected from the group including sulfonic acid, phosphoric acid, nitric acid, perchloric acid, formic acid, acetic acid, propionic acid, succinic acid, gluconic acid, p-hydroxybenzoic acid, salicylic acid, methanesulfonic acid, ethanesulfonic acid, hydroxyethanesulfonic acid, ethylene sulfonic acid, toluene sulfonic acid, naphthyl sulfonic acid, sulfanilic acid, camphorsulfonic acid, quinic acid, o-methylenemandelic acid, hydrogen benzene sulfonic acid and tartaric acid.

6. The method as claimed in claim 2 or 3, wherein the reaction solvent is selected from the group including water, xylene, toluene, tetrahydrofuran, 1,2-dichloroethane, lower alkanol, acetone, ether, dichloromethane, acetonitrile, dimethylsulfoxide, dimethylformamide and a mixture thereof.

7. The method as claimed in claim 2 or 3, wherein the base aqueous solution is at least one selected from the group including a sodium hydroxide aqueous solution, a potassium hydroxide aqueous solution, a potassium carbonate aqueous solution, a calcium carbonate aqueous solution, a sodium methoxide aqueous solution, a sodium hydrogen carbonate aqueous solution, a pyridine aqueous solution, ammonia water, a triethylamine aqueous solution and ethyl diisopropyl amine aqueous solution.

8. The method as claimed in claim 2 or 3, wherein a drying agent is at least one selected from the group including anhydrous magnesium sulfate and anhydrous sodium sulfate.

9. The method as claimed in claim 2 or 3, wherein the crystallization solvent is selected from the group including n-hexane, n-heptane, ethyl acetate, tetrahydrofuran, ether, dichloromethane, chloroform, acetone and a mixture thereof.

10. The method as claimed in claim 2 or 3, wherein, in the cyclization, stirring is carried out at 0 to 150° C. for 1 to 10 hours.

11. The method as claimed in claim 2 or 3, wherein, after the cyclization, the resultant product is cooled to −15 to 50° C.

12. The method as claimed in claim 2 or 3, wherein, in the cyclization, anhydrous sodium acetate is used as a buffering agent.

13. The method as claimed in any one of claims 1 to 3, wherein R in Formula 1 is selected from the group including methyl, ethyl, propyl, isopropyl, butyl, pentyl or hexyl.
